(19) United States
(12) Patent Application Publication
(10) Pub. No.: US 2026/0256603 A1
(43) Pub. Date: Sep. 3, 2026

(54) DEVICES AND METHODS FOR THE TREATMENT OF BENIGN PROSTATIC HYPERPLASIA

(71) Applicant: ELESTA S.P.A., Calenzano (IT)

(72) Inventors: Giovanni MASOTTI, Calenzano (IT); Alessia BLUNDO, Calenzano (IT)

(21) Appl. No.: 19/654,923

(22) Filed: Apr. 22, 2026

Related U.S. Application Data
(63) Continuation-in-part of application No. PCT/EP2024/081845, filed on Nov. 11, 2024.

(30) Foreign Application Priority Data
Nov. 14, 2023 (IT) 102023000024045

(51) Int. Cl. A61F 2/82; A61B 18/14; A61B 18/22; A61B 18/00

(52) U.S. Cl. CPC A61F 2/82; A61B 18/148; A61B 18/22; A61B 2018/00547; A61B 2018/00577; A61F 2210/0004; A61F 2250/0059; A61F 2250/0096

(57) ABSTRACT

Disclosed herein are ablative methods for the treatment of prostate tissues. Tissue ablation, by laser or other suitable energy source, is followed by temporary insertion of a prostatic stent, which is removed or absorbed by the body.

### CROSS REFERENCE TO RELATED APPLICATIONS

This application is a continuation-in-part application of International Application PCT/EP2024/081845, filed November 11, 2024, and claims the benefit of priority under 35 U.S.C. §119 of Italian Application 102023000024045, filed November 14, 2023, the entire contents of which are incorporated herein by reference.

### TECHNICAL FIELD

The present disclosure concerns methods and devices useful for the treatment of benign prostatic hyperplasia in persons affected by lower urinary tract symptoms (LUTS). Disclosed herein are also methods and devices useful for the treatment of different prostate conditions requiring tissue ablation.

### BACKGROUND ART

Benign prostatic hypertrophy (BPH) is a disorder of the prostate that causes increased prostatic volume due to growth of the number of cells in the central part of the prostate gland around the urethra. The data available in literature show an incidence of the disorder between 50-75% in men over the age of 50, which increases to 80% for the over 70s.

BPH presents with obstructive and irritative symptoms and if not treated can lead to complete obstruction of the urinary canal and subsequent need for a catheter to allow emptying of the bladder.

Over the years, many pharmacological therapies and surgical techniques have been developed for the treatment of BPH to reduce the symptoms. Today the gold standard technique is represented by TURP (transurethral resection of the prostate) which is a surgical operation with a high risk of complications including bleeding, infections and impotence.

Alongside the surgical techniques, mini-invasive techniques are being developed which aim to solve the symptoms while reducing the aggressiveness of the approach and the complication rate. This category includes thermal ablation techniques.

Unlike other surgical techniques (e.g. TURP, HoLep, TuLep), thermal ablation techniques (laser, radiofrequency, steam) for the treatment of benign prostatic hyperplasia do not physically remove the tissue treated, but induce a coagulative necrosis in the treated area. In the weeks following the operation this translates into a reduction in volume of the thermally treated tissue via natural resorption processes of the coagulated tissue, therefore restoring the patency of the urethra, which is no longer compressed by the surrounding hypertrophic tissues.

In particular, laser ablation is a mini-invasive procedure that exploits laser energy to produce a local temperature increase which leads to a coagulative necrosis in the area treated and laser-induced cytoreduction (LICR). The applicators are echo-guided into position inside the lesion to be treated, thus obtaining a very precise and controlled localized effect and preserving the surrounding organs and structures. Recently methods have been developed based on the transperineal approach (cf. US 10945789). The resorption times of the necrotic tissue generated by the cell damage caused by the laser radiation vary, but resorption usually begins approximately two weeks after the treatment and reaches maximum effect after three months.

Some ablative techniques based on necrotization of the tissues to be eliminated use cold for similar purposes. In this case it is called cryoablation. The technique entails the application of repeated localized freezing and defrosting cycles.

Current practice after ablative treatment, both laser and using other energy sources, entails the use of a catheter which is left in for a week on average to facilitate urination of the patient while waiting for the pressure on the urethra to start decreasing as a result of the ablative treatment. After the ablative treatment, a local oedema is created which leads to an increase in the lower urinary tract symptoms (LUTS) on the days immediately following the treatment, but then gradually diminishing until complete disappearance of the compressive symptoms. The purpose of the catheter, in the most critical cases, is to maintain the canal open until decompression induced by the ablative treatment.

Catheterization undoubtedly causes patient discomfort and the transition phase, which initially entails a temporary worsening of the symptoms, can in some cases discourage the patient and lead him to choose solutions that are more invasive but promise immediate effects. The mini-invasive techniques and in particular laser ablation with transperineal approach are promising because they allow a reduction in LUTS while preserving the functions of the organ and they have a very low complication rate and minimum side effects. Today the transition phase for inducing resorption of the tissue treated, therefore requiring catheterization in many cases, could be factors limiting the use and expansion of the technique, leading many patients to choose more invasive treatments with many more contraindications and complications to obtain an immediate effect.

The need for a catheter and a transition phase before disappearance of the symptoms is, in fact, a psychological aspect not to be underestimated in patient acceptance of the method.

An object of the disclosure disclosed here is to solve or alleviate the problems connected with the mini-invasive treatment of benign prostatic hyperplasia (BPH) by means of ablative methods. Another object of the disclosure is to provide a method for treatment of a prostate condition requiring tissue ablation, other than BPH, for instance to remove cancerous tissue.

### SUMMARY

To reduce patient discomfort due to the need for the use of temporary catheters while awaiting reduction of the compression of the urethra following ablation treatment, and to facilitate large-scale development and diffusion of the mini-invasive method, the use of a prostatic stent is proposed, to be inserted in the urethra, in the section that passes through the prostate, after ablative treatment. The stent acts as a bridge, i.e. as a temporary aid designed to maintain the patency of the urethra, until the ablative procedure has achieved its full effect. Essentially, the stent maintains the urethra dilated during the phase after the laser ablation (or ablation with another energy source) while awaiting reduction of the volume of the prostate gland resulting from the ablation treatment. The stent enables the patient to return to his normal life immediately after the treatment, without requiring the use of a catheter, albeit temporary.

Advantageously, according to an aspect disclosed here, the stent comprises a plurality of annular elements joined to one another by a longitudinal structure.

Compared to other prior art stents such as, for example, those disclosed in US2011/301259, WO02/098476 and US2021/059704, the structure with annular elements connected by a longitudinal structure offers multiple advantages.

The geometry with annular elements joined to one another simplifies the manufacturing process since the geometries of the prior art stents, disclosed for example in the two documents mentioned above, have a more complex geometry and require a polymer fibre extrusion process. The geometry described here, vice versa, can be reproduced by moulding.

Furthermore, the geometry with annular elements reduces the cell growth inside the stent, which can occur with filament structures as described in the prior publications cited above.

Compared to US2021/00597004, a geometry with annular elements does not create incisions in the muscle of the bladder neck or urethra.

To increase acceptability of the stent (or dilator) and not jeopardise the benefits of the mini-invasive ablation technique, in some embodiments it is advantageous for the stent itself to be minimally invasive.

A reduction in the invasiveness of the stent can be advantageously obtained during the removal phase, for example, by producing the stent in biodegradable and/or bioresorbable material. Biodegradability of the device obviates the need for further operations to remove it. Essentially, the stent can be made of a material that maintains sufficient compression resistance for the time necessary for reduction of the volume of the prostate gland following the ablation procedure. For example, the stent can be made of a material that guarantees a variable duration from two to four weeks. The degradation or bioresorption allow elimination of the stent without removal operations.

The stent can be made for example of a material selected from the group comprising: polylactic acid (PLA), polyglycolic acid (PGA), polycaprolactone (PCL), polydioxanone (PDO), and copolymers thereof.

In some embodiments, the stent can be configured to withstand pressures in the range of 1-10 kPa, for example. If the stent is made of biodegradable or bioresorbable material, the above-mentioned materials have suitable mechanical characteristics and can reach rupture pressure values in the order of several Mpa.

Alternatively to a stent made of biodegradable material, it is possible to produce the stent with a structure that makes removal easy without the need for a post-operative examination and if possible without accessories that remain in position in the urethra and protrude from it to allow removal of the stent, since said accessories can cause patient discomfort and carry infections of the lower urinary tract.

In some embodiments, with a biodegradable or bioresorbable stent, or also with a non-biodegradable or non-bioresorbable stent, the stent can be provided with an internal sensor adapted to provide information on the oedema condition and evaluate when the stent can be removed in order to avoid the need to insert a new one in the case of premature removal, i.e. before patency of the canal has been restored.

In some embodiments, the stent can comprise one or more pressure sensors applied along the longitudinal extension, adapted to detect the pressure exerted on the stent by the surrounding tissues. These sensors are able to provide data indicative of the pressure reduction exerted by the tissues of the treated prostate gland; said reduction takes place over time due to the natural resorption processes of the coagulated tissue. The pressure sensor signal can be compared with a threshold value below which the tissues can be considered sufficiently reabsorbed to allow removal of the stent without entailing compression of the urethra by the surrounding tissues.

In other embodiments, the stent can be provided with one or more biochemical sensors adapted to detect the presence of a substance indicative of oedema and inflammation or adapted to detect variations in the pH. In this case the signal provided by the sensor(s) indicates when the concentration of the substance indicative of the presence of oedema and inflammation drops below a threshold, which indicates that the oedema has reduced. For example, the sensors can be adapted to detect the concentration of macrophages or the presence of DAMPs (damage-associated molecular patterns). In general, they are components that can be found in urine in the case of inflammatory processes in progress. In this case, it is expedient for the sensor to be positioned on the inner side of the stent. In other cases, the components detectable by the sensor can be components produced by the tissue treated, in which case it is expedient for the sensor to be positioned on the outer side of the stent. Examples of sensors that can be used are described in: Shuobo Shi, Ee Lui Ang, Huimin Zhao: “In vivo biosensors: mechanisms, development, and applications”, Journal of Industrial Microbiology and Biotechnology, Vol. Volume 45, Issue 7, 1 July 2018, Pages 491–516, DOI:10.1007/s10295-018-2004; Vadgama, P, “Monitoring with In Vivo Electrochemical Sensors: Navigating the Complexities of Blood and Tissue Reactivity”, in Sensors, 2020. DOI: 10.3390/s20113149; Macovei, DG., Irimes, MB., Hosu, O. et al. “Point-of-care electrochemical testing of biomarkers involved in inflammatory and inflammatory-associated medical conditions”, in Anal. Bioanal. Chem, Vol. 415, 1033–1063 (2023). DOI:10.1007/s00216-022-04320-z.

In some embodiments passive sensors are used interrogated from the outside, for example by: radiofrequency, RFID, ultrasound, optical energy or wireless modules. Examples of sensors of this type are described in: Di Lu, Ying Yan, Yujun Deng, Quansan Yang, Jie Zhao, Min-Ho Seo, Wubin Bai, Matthew R. MacEwan, Yonggang Huang, Wilson Z. Ray, John A. Rogers “Bioresorbable Wireless Sensors as Temporary Implants for In Vivo Measurements of Pressure”, in Advanced Functional Materials, 2020. DOI:10.1002/adfm.202003754;Rahul Singh, Mohammad Javad Bathaei, Emin Istif, and Levent Beker “A Review of Bioresorbable Implantable Medical Devices: Materials, Fabrication, and Implementation”, in Adv. Healthcare Mater, 2020, Vol. 2000790. DOI: 10.1002/adhm.202000790.

In some embodiments, with particular advantage if the stent is bioresorbable or biodegradable, the sensors could also be biodegradable or bioresorbable and made, for example, of inorganic material (e.g.: silicon (Si), germanium (Ge), silicon-germanium (SiGe), zinc or zinc oxide (ZnO), magnesium (Mg), silica (SiO2)) and organic material (e.g: polylactic glycolic acid (PLGA) or polycaprolactone (PCL) or silicones). Examples of these types of devices are described in: Wei, Z., Xue, Z. e Guo, Q.: “Recent Progress on Bioresorbable Passive Electronic Devices and Systems”; Micromachines, 2021, Vol. 12,600. DOI:10.3390/mi12060600; A. A. La Mattina, Dr. S. Mariani, Dr. G. Barillaro: “Bioresorbable Materials on the Rise: From Electronic Components and Physical Sensor to In Vivo Monitoring Systems”, ADVANCED SCIENCE, 2020, Vol. 7, 1902872. DOI: 10.1002/advs.201902872.

In some embodiments, the signals received from the sensors could be used to activate the release in situ of medicines or other drugs, possibly by wireless activation.

When the stent is provided with one or more sensors, adapted to detect parameters indicative of the state of reduction of the oedema or other clinical conditions indicative of the post-operative recovery phase, the datum or data provided by the sensors can be used in a system of artificial intelligence or predictive analysis of the evolution of the patient’s condition. It is possible, for example, to collect data from sensors on already implanted stents and correlate with this data information on the clinical condition of the patient and/or evolution of the post-operative phase. Once enough data have been collected and classified, it is possible to make predictive evaluations on the evolution of the clinical situation of a new patient based on the data provided by the sensor(s) arranged on the stent. These data are used to interrogate the database and obtain from said data information on the post-operative recovery phase. This can be useful for informing the patient, for example, of how many days it is estimated will be needed for complete recovery, or for the stent to completely dissolve (when bio-resorbable) or for it to be removed (when not bio-absorbable). Via a comparison between the data collected and the measurements provided by the sensors, it is also possible to indicate to the patient if and in how many days’ time a check-up is advisable. This allows the frequency of check-ups to be optimized and/or unnecessary check-ups to be avoided.

In some embodiments, a magnetic mechanism can be used to remove the stent. In this case the stent can be provided with a ferro-magnetic element or component, which interacts with an external magnetic extractor, adapted to magnetically hook and remove the stent. Advantageously, in some embodiments the external magnet can have an annular shape to prevent the stent exerting a crushing force on the urethra wall.

Furthermore, to guarantee reduced invasiveness, the stent can be configured to maintain the physiological condition of contact between urinary flow and epithelial wall of the urethra. For said purpose the stent can include openings or windows along the extension thereof, as illustrated below with reference to exemplary embodiments.

In some embodiments, to increase the biocompatibility and ease of insertion, the stent can be provided with a coating with biocompatible or anaesthetic material, or containing anti-inflammatory or antibiotic drugs or combinations thereof, also to reduce the risk of infections of the lower urinary tract.

The material used to produce the stent can furthermore be customised to guarantee a degradation rate compatible with clinical requirements which could translate into different models of the device. For example, the component material of the stent can have a formulation chosen as a function of the required degradation time, as a function of the time interval which it is estimated must elapse with the stent implanted and working efficiently to achieve patency of the urethra after the ablation treatment.

To facilitate insertion, the stent can have, in cross section, an interrupted annular development. In practice, in this case the stent has a spiral cross section, the width of which can be reduced to facilitate insertion. A characteristic of elastic deformability of the stent allows the subsequent expansion thereof to the original dimension once positioned inside the urethra. Essentially, the stent can be closed by coiling up and can then open once in position. The insertion can be facilitated via the use of a small diameter tubular insertion device, in which the stent is inserted after coiling up, thus ensuring sufficiently limited dimensions for insertion into the insertion device.

To facilitate insertion, the stent can for example be inserted into and then subsequently expanded in situ, once the final position has been reached with saline solution or other sterile solution or hydrogel.

According to further aspects disclosed herein, a method for treating a prostate condition by combining an ablative intervention on prostate tissue with temporary stenting of the prostatic urethra is disclosed. The temporary stent is placed after the ablative intervention and remains in place during a post-treatment period sufficient to maintain urethral patency while edema subsides, necrotic tissue is resorbed, treated tissue contracts, or obstructive tissue is otherwise reduced. The temporary stent can be removable or bioabsorbable. In this manner, the patient can benefit from the tissue-reduction effect of the ablative intervention while also obtaining immediate or early maintenance of urethral patency.

In some embodiments, the prostate condition treated can be benign prostatic hyperplasia, prostatomegaly, obstructive median lobe enlargement, prostate cancer, a focal prostate lesion, or another condition in which reduction, destruction, necrosis, debulking, or removal of prostate tissue is clinically useful.

In some embodiments, the ablative intervention can be performed by delivery of energy to target prostate tissue. The energy can comprise laser energy, radiofrequency energy, microwave energy, ultrasound energy, electrosurgical energy, thermal energy delivered by steam or heated fluid, cryogenic energy, irreversible electroporation, or combinations thereof. In some embodiments, the energy causes coagulative necrosis, thermal injury, freezing injury, electrical injury, or another tissue-destructive effect resulting in subsequent reduction of tissue volume or reduction of urethral compression.

In some embodiments, the ablative intervention can be carried out through the urethra, transperineally, transrectally, transdermally, or by another minimally invasive route suitable for delivering energy into the target prostate tissue. In transurethral embodiments, the target tissue can be treated using an endoscope, cystoscope, resectoscope, catheter, steerable delivery device, or another transurethral access instrument. In transperineal embodiments, the target tissue can be treated using needles, cannulas, probes, optical fibers, electrodes, or other applicators introduced through the perineum.

In some embodiments, one or more treatment members are positioned adjacent or inside target prostate tissue and activated to treat one or more tissue volumes. The treatment can be focal, multifocal, lobar, circumferential, sectoral, or whole-gland.

In some embodiments, the treatment members are repositioned sequentially, for example in a pull-back technique, to treat multiple tissue volumes. In other embodiments, a plurality of treatment members are operated simultaneously.

In some embodiments, after the ablative intervention, a temporary prostatic stent is inserted into the urethra in the portion extending through the prostate. The stent acts as a temporary bridge to maintain lumen patency during the recovery phase after the ablative intervention. The stent can be maintained until the prostate tissue has been sufficiently reduced, resorbed, contracted, or remodeled so that the urethra remains patent without the stent. The stent can be removed, or, if made of bioabsorbable or bioresorbable material, allowed to degrade in vivo, after a time span.

In some embodiments, the temporary prostatic stent can be any stent suitable for maintaining patency of the prostatic urethra during the post-treatment interval. The stent can be expandable, self-expanding, resiliently deformable, compressible for delivery, and/or expandable in situ. The stent can comprise annular elements joined by one or more longitudinal structures, can have an open-loop cross section, can comprise openings or windows allowing urinary flow to contact the urethral wall, and can include anchoring structures that resist migration. In some embodiments, the stent can comprise a magnetic element to facilitate magnetic extraction. In some embodiments, the stent can include a coating comprising a lubricious, biocompatible, anesthetic, anti-inflammatory, antimicrobial, antibiotic, antiproliferative, or drug-eluting material.

In some embodiments, the temporary prostatic stent can comprise one or more sensors configured to detect pressure, flow, edema, inflammation, pH, biochemical markers, tissue response, or another parameter indicative of recovery after treatment. Sensor data can be used to decide when the stent should be removed or to monitor the post-treatment course.

In some embodiments, the stent can remain implanted for hours, days, or weeks. In some embodiments, the stent remains implanted for a period from about 1 day to about 8 weeks, for example from about 1 week to about 4 weeks. In some embodiments, the selected duration depends on the modality of tissue treatment, the amount of tissue treated, the degree of expected edema, the patient anatomy, the pathology being treated, or the predicted recovery time.

In some embodiments, the temporary stent is used following treatment that does not immediately create a large open cavity in the urethral channel but instead produces delayed decompression, delayed tissue shrinkage, delayed necrotic resorption, or transient post-operative swelling. The temporary stent can therefore complement the ablative intervention by preserving urinary function during the interval between treatment and durable decompression.

### BRIEF DESCRIPTION OF THE DRAWINGS

The invention will be better understood by following the description and the attached drawings, which show illustrative non-limiting embodiments of the invention. More specifically, the drawings show:

FIG. 1 is a lateral view of a stent in a lateral view, in one embodiment;

FIG. 2 is a lateral view of a stent in a lateral view, in a further embodiment;

FIG. 3 is a cross section of the stent of FIG. 2;

FIG. 4 is a cross section of the stent of FIG. 2 in a modified embodiment;

FIG. 5 is a longitudinal section of a stent, in a further embodiment;

FIG. 6 is a lateral view of a stent, in a further embodiment;

FIG. 7 is a cross section of a stent in a further embodiment;

FIG. 8 and 9 are a cross section of the stent of FIG. 7 in two different conditions of use;

FIG. 10 is a schematic view of a stent applied to a patient;

FIG. 11, 12 and 13 are schematic sections of an operation area according to a method described herein;

FIG. 14A and 14B are cross sections of the prostate gland with indication of the operation areas;

FIG. 15 is a section analogous to the section of FIG. 14B after the insertion of a stent;

FIG. 16 is an anteroposterior section of the operation area after application of the stent;

FIG. 17 is a section analogous to the section of FIG. 14A, 14B and 15 after removal of the stent;

FIG. 18(A)-18(D) are extraction phases of a stent by means of magnetic extractor;

FIG. 19 are figures of magnets for magnetic extractors;

FIG. 20 and 21 are figures of stents with magnets adapted to cooperate with an external extractor.

### DETAILED DESCRIPTION

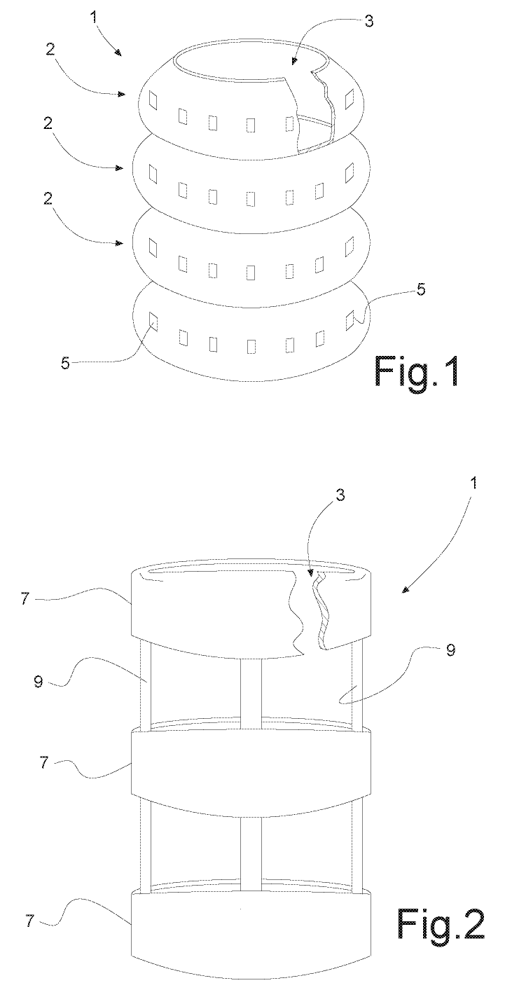

FIG. 1 shows a lateral view of a stent 1 according to the present disclosure in one embodiment. The stent 1 comprises a body consisting of a sequence of hollow components with toroidal wall 2, joined to one another to form a single body with a longitudinal extension. The number 3 indicates a flow channel. In embodiments, the toroidal walls of the single components can be provided with openings or windows 5, so that once the stent has been implanted, the urinary flow can bathe the epithelium of the urethra, and maintain the normal physiological condition of the latter.

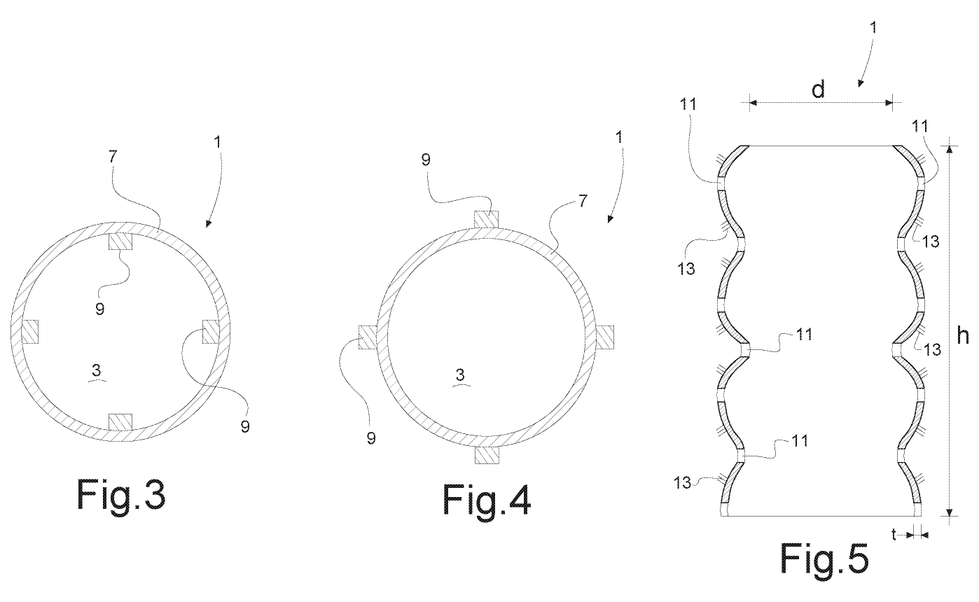

FIG. 2 shows a further embodiment of a stent, again indicated overall by the number 1. In this embodiment, the stent 1 comprises a plurality of annular components 7 with cylindrical extension, joined to one another by longitudinal structures 9. These longitudinal structures 9 can consist of linear ribs positioned inside the annular components 7, as shown in the section of FIG. 3. In other embodiments, the longitudinal structure 9 can comprise ribs positioned on the outside of the annular components 7, as shown by way of example in FIG. 4. The assembly of annular components 7 and longitudinal structures 9 define an open grid structure, with large openings that allow the urinary flow to bathe the epithelium of the urethra, when the stent is implanted. The number 3 again indicates the flow channel for the passage of the urinary flow.

FIG. 5 shows a longitudinal section of a further embodiment of a stent 1 provided with a toroidal wall having undulated longitudinal section. In the embodiment of FIG. 5 the stent has a gradually increasing cross section from a first end (proximal when the stent is installed) to a second end (distal). The number 11 indicates openings or windows made in the undulated wall of the stent, to allow the flow of urine, which flows in the flow passage 3, to bathe the epithelium of the urethra. The dimensions h (longitudinal length), d (diameter) and t (thickness) shown in FIG. 5 can have, by way of example, the following values:

h between 40 and 90 mm

d between 5 and 10 mm

t between 0.1 and 2 mm or between 0.5 and 2 mm.

The other embodiments described herein may have analogous dimensions.

In the embodiment of FIG. 5 on the outer surface of the stent 1, anchoring elements or structures are provided for anchoring to the urethra wall. In FIG. 5 these structures are schematically indicated by the number 13 and can for example comprise cilia, lamellae, protuberances or other members protruding radially from the stent 1. In some embodiments, the anchoring structures 13 are inclined with respect to the radial direction, to perform a retaining and anchoring function both in the direction of the urinary flow and in the opposite direction, thus guaranteeing retention of the stent 1 in the correct position inside the urethra. If the stent 1 is made of bioresorbable material, the anchoring in the urethra does not constitute an impediment to its removal, since the stent is removed by dissolving over time. In other cases, if the stent has to be removable, its shape can be such as to allow a reduction of the diameter, as described herein for other embodiments, so as to release the anchoring structures 13 from the urethra wall.

FIG. 6 shows a lateral view of a stent, again indicated by the number 1, in a further embodiment. Also in this case the stent 1 comprises a flow channel 3, defined inside a structure formed of annular elements 15 joined to one another by longitudinal structures 17. In the embodiment shown in FIG. 6 the annular elements 15 have a toroidal outer surface and the longitudinal structures 17 can be undulated. In other embodiments, the longitudinal structures 17, which can consist of, or comprise, ribs extending from one to the other of the annular elements 15, can be rectilinear instead of undulated. Also in this embodiment the stent 1 has large openings to allow the urinary flow to bathe the epithelium of the urethra. In FIG. 6 the stent 1 is shown equipped with anchoring structures 19, analogous to the anchoring structures 13 of FIG. 5.

Anchoring structures analogous to the structures 13 and 19 can be provided also in the other stent illustrated herein.

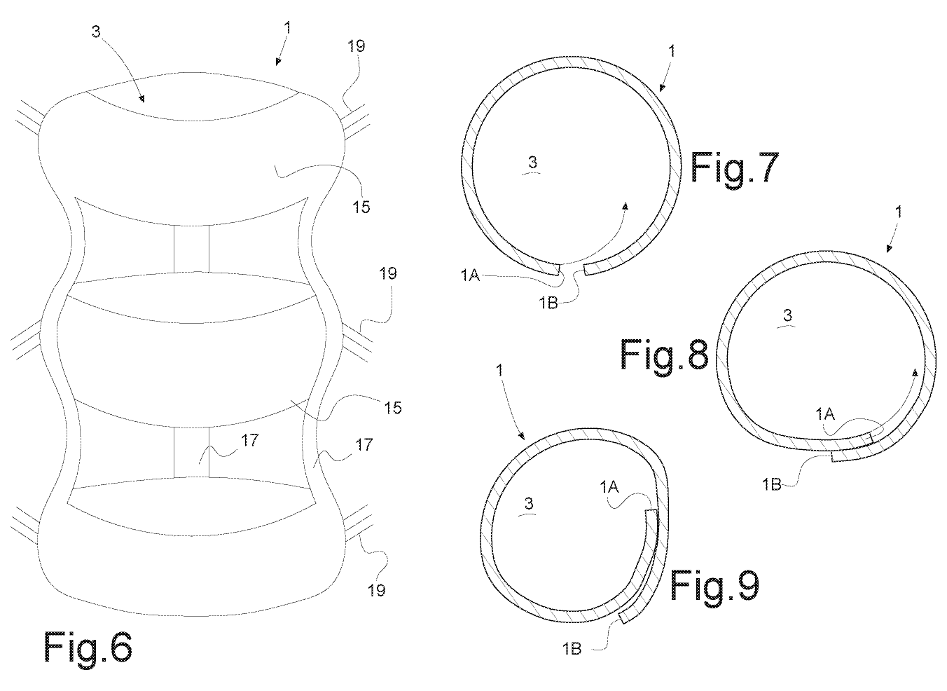

In some embodiments, the stent 1 can be configured to allow a variation in its diameter, namely in its cross section, to facilitate insertion into position in the patient’s urethra. For said purpose, the stent can be made for example of an annularly discontinuous structure, namely having a cut or interruption that extends longitudinally for the entire length extension of the stent 1. An embodiment of this type is illustrated, for example, in FIG. 7, 8 and 9. For simplicity, these figures show only the cross section of the stent 1. In FIG. 7 the stent is shown in a rest condition, namely not implanted in the urethra. In the cross-sectional view the stent has an interrupted annular shape. The numbers 1A and 1B indicate two longitudinal edges that define an interruption extending for the entire length of the stent 1. In this way, as can be seen in FIG. 8 and 9, the diametral dimension of the stent can be reduced, by coiling the cross section thereof, with one of the longitudinal edges 1B inserted below the other longitudinal edge 1A.

In FIG. 8 the stent is compressed until it has a diametral dimension smaller than the dimension at rest and such as to facilitate insertion into the urethra. This insertion can be facilitated by the use of the tubular instrument, which has an external diameter compatible with the dimension of the urethra and an internal diameter sufficient to accommodate the stent when diametrally compressed (FIG. 8). The instrument with the stent inside can be inserted into the urethra as far as the position in which the stent 1 is to be applied. Subsequently, the instrument is retracted, maintaining the stent in position, for example by means of a cannula or other linear member inserted in the stent insertion instrument. The relative movement between cannula and insertion instrument causes the stent to project from the instrument. The extracted stent dilates until reaching the position of FIG. 7, or an intermediate position (FIG. 9).

A longitudinal interruption as shown in the cross sections of FIG. 7, 8 and 9 can be provided in each of the embodiments described above.

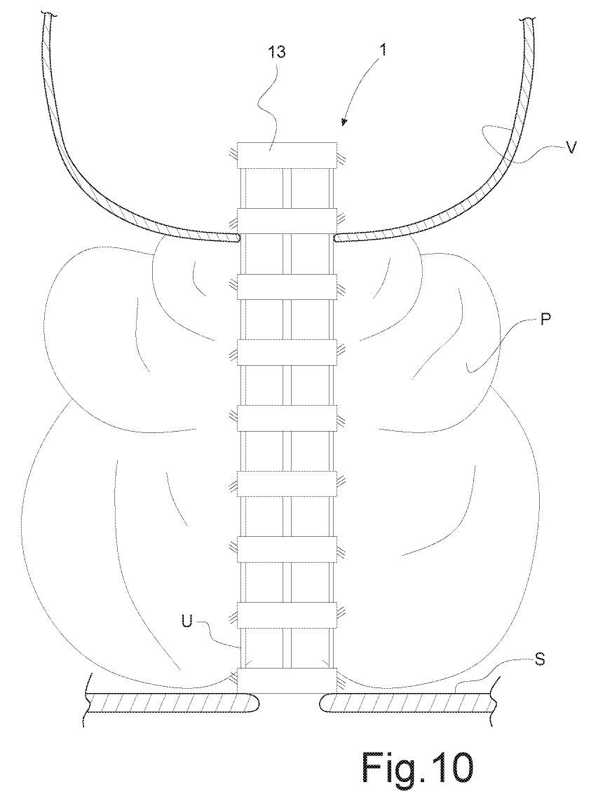

FIG. 10 shows schematically a stent 1 inserted in position in the urethra U of a patient. In FIG. 10 the following anatomical parts are shown, in addition to the urethra U: the bladder V, the sphincter S, the prostate P. The stent 1 is inserted deep in the urethra and has a length such as to involve the entire area of the urethra U surrounded by the prostate P. The proximal end of the stent 1 can protrude into the bladder V. The presence of openings in the stent, as described above, allows (in addition to bathing the epithelium of the urethra with the urinary flow) retention of urine in the bladder V to be avoided, also when the proximal end of the stent 1 protrudes into the bladder.

The stent 1 described so far can be used as a medical device in surgical operations to reduce the volume of the prostate gland P via the use of any method suitable for the purpose. In general, however, the stent 1 can also be used in combination with a cryoablation method, and in embodiments described here in further detail, the ablation is performed via the contribution of energy which, interacting with the tissues, is transformed into thermal energy. In general, a form of energy in radiofrequency can be used, or also a steam flow. Below, however, the ablation procedure will be described specifically with reference to the use of laser radiation from one or more sources. Although currently preferred in some respects, this should be understood as a non-limiting example.

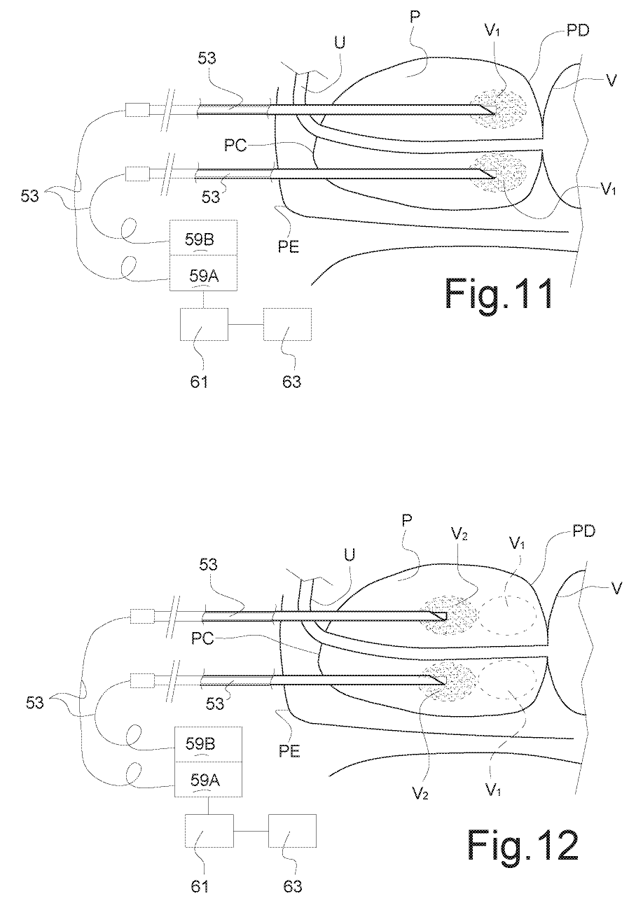

FIG. 11 to 17 show schematically various phases of the procedure for the treatment of prostatic hyperplasia with the use of a stent as described here.

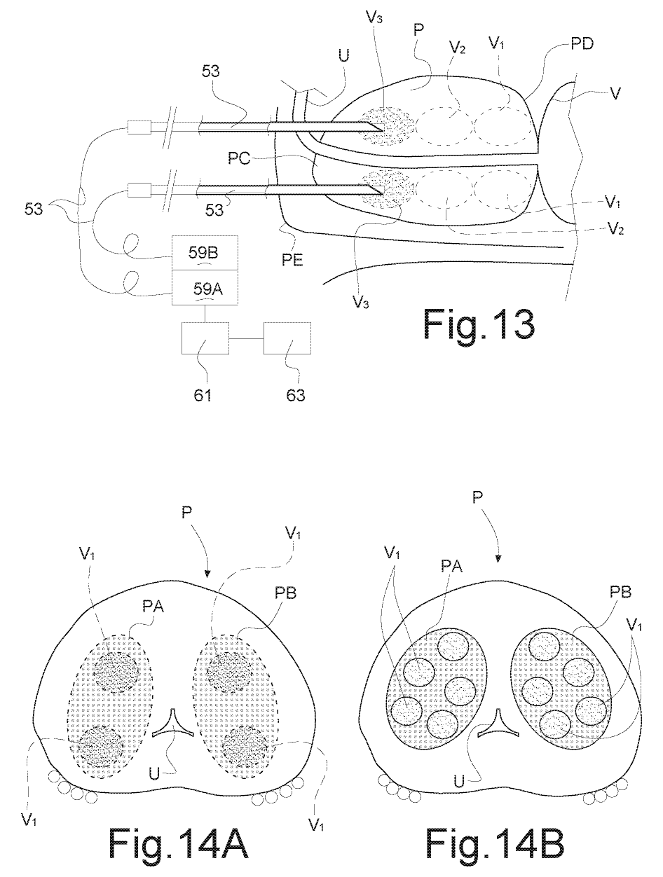

The laser treatment can be carried out by inserting one or more hollow needles or insertion devices in both lobes PA, PB (FIG. 14A, 14B) of the prostate P and introducing an optical fibre through each needle, so that laser energy can be delivered into the adenomatous tissue causing necrosis and subsequent resorption. The two lobes can be treated simultaneously. In other embodiments, each lobe can be treated separately from the other, namely the two lobes can be treated in sequence.

The needles and the optical fibres are inserted through the perineum, namely transperineally. The number of needles inserted in each lobe of the prostate P can depend on the dimension of the prostate P and the quantity of adenomatous tissue to be removed. Two or more needles or insertion devices can be inserted simultaneously in each lobe PA, PB of the prostate P, for example with the aid of a guide grid (not shown), so that volumes of adjacent or nearby prostatic tissue can be treated simultaneously. In other embodiments, one or more needles or insertion devices can be inserted in sequence in the prostatic tissue, to treat nearby or adjacent volumes of the adenoma at different times. This second approach will require a longer treatment time.

The number of needles introduced simultaneously can depend, among other things, on the number of laser sources available. It may be advantageousto provide as many independent laser sources as optical fibres functioning simultaneously.

During the treatment, one or more needles or insertion devices and relative optical fibres can be moved along the axis of the needle so that successive volumes of tissue can be irradiated with laser radiation in a pull-back procedure. FIG. 11, 12 and 13 show in a section view according to a sagittal plane three different laser treatment phases of the prostate P. In the embodiment example illustrated, two insertion devices or needles 51, each of which guides a respective optical fibre 53, are inserted into each lobe PA, PB of the prostate P via the transperineal route. If a third lobe is present, it can be treated by appropriate insertion and positioning of the laser optical fibre (not shown). The needles 51 are inserted through the perineum PE, namely through the area between the scrotum (not shown) and the anus A of the patient.

According to the embodiment shown in FIG. 11 to 17, each needle or insertion device 51 and relative optical fibre 53 are completely inserted into the prostate P as far as the position of FIG. 11, where the treatment will begin. This is the position in which the tips of the needle 51 are farthest from the apex PC of the prostate P and closer to the base of the prostate PD and to the floor of the bladder V. The laser energy generated by a laser source is conveyed through the optical fibres 53 to the tips thereof, which are positioned at or near the tips of the needles 51, or can protrude from said tips.

According to some embodiments, independent laser sources can be provided for different optical fibres. FIG. 11 shows a laser source 59A, 59B for each optical fibre 53. Each laser source can be controlled independently of the others so that, for example, each laser source can be switched on or off and its laser emission can be adjusted independently of the other sources. For example, the emission power, emission time, energy dose and pulse frequency (in the case of pulsed laser) can be adjusted independently for each source.

In other embodiments each source can be coupled with several optical fibres.

In some embodiments it is possible to use, also in combination, different laser frequencies, namely sources that emit at different wavelengths.

Insertion of the needles or insertion devices 51 and optical fibres 53, and the subsequent movement thereof in the prostate, can be carried out with the aid of ultrasounds (US) using an echographic probe, for example an endorectal probe or a probe resting directly on the perineum, not shown. In other embodiments, the needles can be inserted by means of magnetic resonance in combination with insertion devices or amagnetic needles, or using any other suitable imaging method, including systems for the fusion of images obtained via magnetic resonance and ultrasounds, for greater accuracy in positioning of the applicators.

The laser emission can be controlled by a control unit 61, which can be functionally connected to the laser sources 59A, 59B and to a user interface 63. A controlled dose of laser energy is delivered by the laser source(s) through the optical fibres 53 to cause denaturation and necrosis of the tissue in a volume surrounding the tip of the optical fibre and/or in front of said tip. In FIG. 11, V1 indicates the volume of adenomatous tissue that can be treated by the laser while the tip of the optical fibre 53 is kept in the position of FIG. 11.

To treat a lager quantity of tissue, the optical fibre 53 and the relative hollow needle or insertion device 51 can be gradually moved outside the body of the patient. For example, once the volume of tissue V1 has been treated by the laser energy delivered through the optical fibres 53 in the first position of FIG. 11, the needles 51 and the relative optical fibres 53 housed therein can be extracted gradually in the direction f11, as far as the position of FIG. 12. The hollow needles 51 and the optical fibres 53 can then be kept in the new position of FIG. 12 for a given period of time, during which the laser radiation generated by the laser sources 59A, 59B irradiates the tissue in the volume V2. Once the tissue in the volume V2 has been treated, the needles 51 and the optical fibres 53 are moved a further step towards the outside, until they reach the position of FIG. 13, where a third volume of adenomatous tissue V3 is treated by each optical fibre 53.

As can be seen from FIG. 13, in a three-phase process, two elongated volumes of adenomatous tissue have been treated by laser radiation along the pull-back movement trajectory of the two hollow needles 51 and relative optical fibres 53 in the lobe PA. The same operation can be performed simultaneously or subsequently in the lobe PB, so that at the end of the process four volumes of tissue have been treated by means of laser radiation, around the urethra U. These four volumes are shown in cross section in FIG. 14A and generically indicated therein by V.

The number of needles and fibres for each lobe PA, PB of the prostate P can be lower or higher than the number indicated above. FIG. 14B shows the situation obtained by treating each lobe with five needles and five fibres, obtaining five volumes treated, again indicated by V.

The tissue treated by delivery of energy, which leads to denaturation and necrosis of the tissue, will be gradually eliminated by the natural phenomena of absorption activated by the organism, thus obtaining the desired end result, namely reduction of the total volume of tissue of the prostate gland and lightening or reduction of the compression on the urethra.

However, in the phase immediately after the delivery of energy and treatment of the tissues, reduction of the urethra compression is not obtained. On the contrary, the oedema resulting from the treatment may tend to increase the urethra compression. In FIG. 14A and 14B this condition is represented by the fact that the urethra U is shown still compressed and the overall volume of the prostate tissue has not diminished.

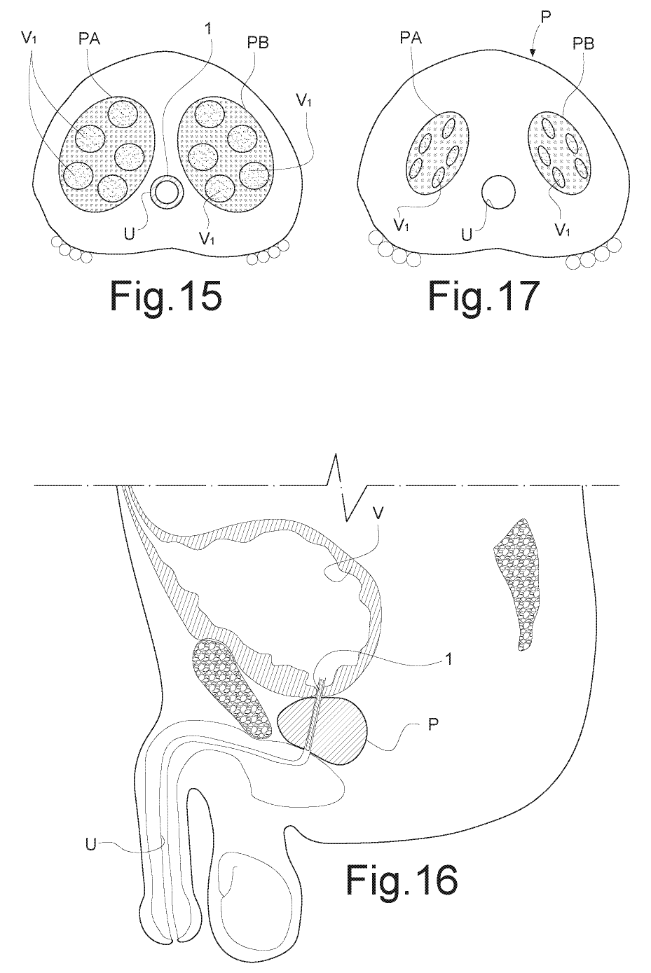

In order to allow the patient to obtain immediate relief and resume normal urethra functionality, according to the method described here, once the denaturation treatment of the tissue in the volumes V has been carried out, in the urethra U a stent 1 of the type described above is inserted, as indicated in FIG. 15. The position of the stent 1 can be seen better in the section in the anteroposterior plane shown in FIG. 16.

The treated tissue, denatured and/or necrotized, is resorbed in a period of time that can vary typically from two to four weeks. The stent 1 must remain in position preferably for the entire interval of time necessary for complete resorption of the treated tissue, so as to maintain the patency of the urethra U until complete decompression obtained by reduction of the treated tissue volumes.

FIG. 17 shows the prostate P with reduced volume, as it appears after resorption of the treated tissues. The stent 1 has been removed from the urethra U which, thanks to the reduction in volume of the prostate gland P, is naturally patent.

The stent 1 is preferably removed, as mentioned, by resorption, thanks to the use of bio-resorbable materials for production of the stent. This avoids the need for a medical examination for removal of the stent and increases patient comfort.

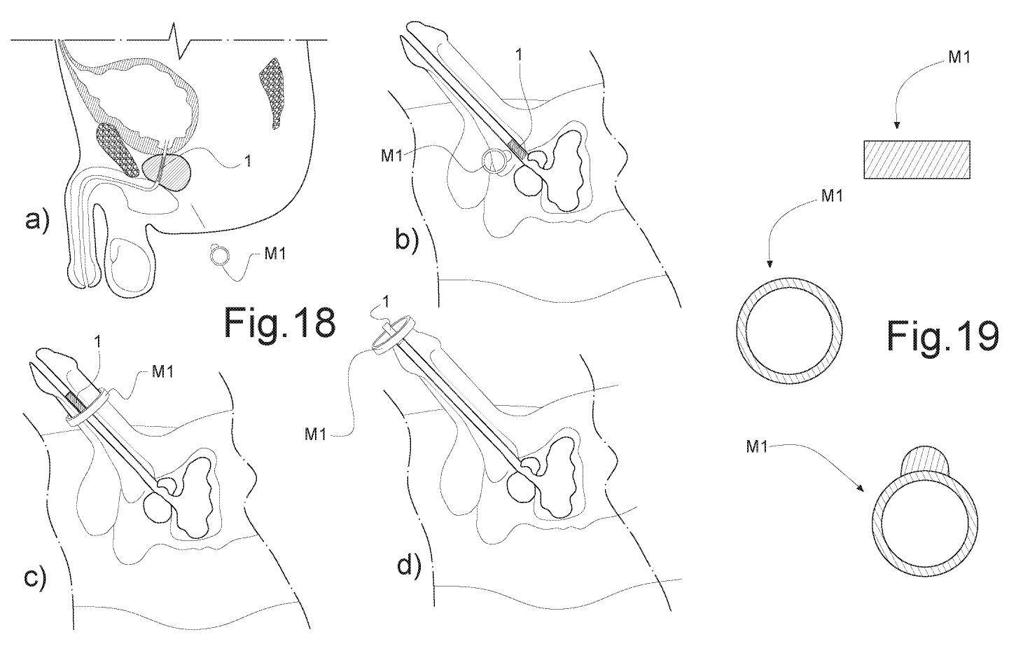

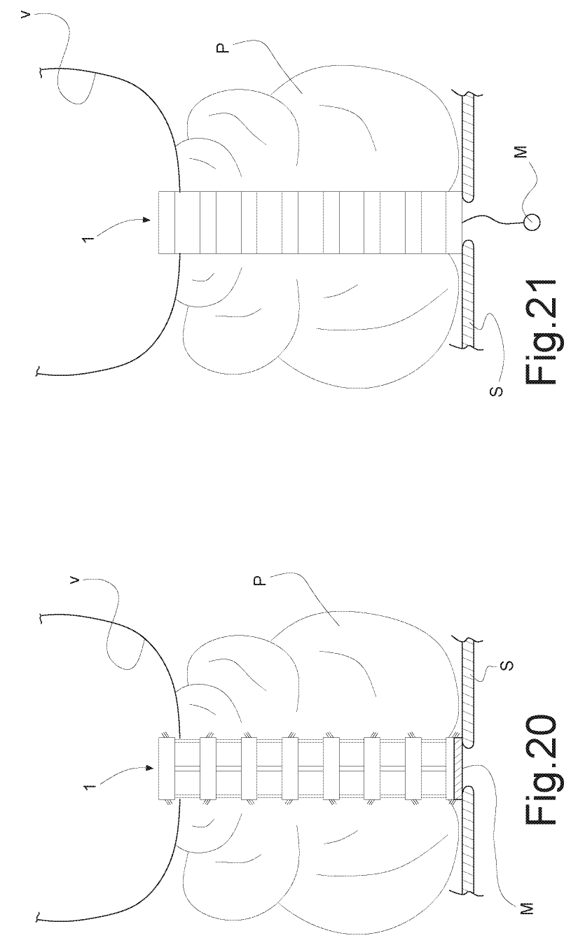

Alternatively, a removal system by means of an external device, typically magnetic, which cooperates with a magnet M integral with the stent 1 can be provided. FIG. 18(A)-18(D) show, for example, an embodiment in which by means of an external magnetic extractor M1, a stent 1 provided with a permanent magnet M is extracted. The extractor can be provided if necessary with an annular-shaped magnet M1, as shown schematically in FIG. 18(A)-18(D), which illustrate a removal sequence. Preferably, to adapt to the morphology of the area in which the stent 21 is applied, two extractors with magnets of different shapes can be used: an annular-shaped magnet M1 to complete (FIG. 18(C), 18(D)) the extraction of the stent 1 along the part of the urethra extending through the external genitals, while the initial part of the extraction (FIG. 18(A), 18(B)) from the inner area of the urethra can be initiated by a magnet having another shape, for example prismatic. Possible shapes of external magnets suitable for the purpose are shown in FIG. 19. FIG. 20 and 21 show embodiments of internal magnets M, fixed to the stent 1. In FIG. 20 the magnet M is integrated in the stent 1, while in FIG. 21 it is attached to the stent 1 by means of a wire connection or similar.

The post-operative recovery phase can be facilitated, and patient comfort can be further improved, by providing for the stent to have a coating consisting of, or containing, one or more drugs aimed at reducing the inflammation and/or preventing infections. For example, an external coating of the stent 1 can comprise an anaesthetic drug, an anti-inflammatory drug, an antibiotic drug or combinations thereof.

The above description illustrates embodiments of stents according to the present disclosure, the scope of which is defined by the following claims.

In addition to the innovative characteristics of the prostatic stents described above and defined in the attached claims, the following also form subjects of the present disclosure:

Clause 1. A method for the treatment of benign prostatic hyperplasia, comprising the step of inserting a prostatic stent into the urethra of a patient on whom a mini-invasive operation of ablation of the prostatic tissues has been performed, and in which the stent is kept in place for a time sufficient to reduce the compression of the urethra by the surrounding prostatic tissues, previously subjected to ablative treatment, the reduction of the compression resulting from resorption processes of said tissues.

Clause 2. The method of clause 1, wherein the ablative treatment is a transperineal treatment.

Clause 3. The method of clause 1 or 2, wherein the ablative treatment is a treatment performed by means of contribution of energy.

Clause 4. The method of clause 3, wherein the ablative treatment is a treatment performed by means of delivery of a laser radiation.

Clause 5. The method of any one of the clauses 1 to 4, wherein the stent is made of bioresorbable or biodegradable material.

Clause 6. The method of any one of the claims from 1 to 4, furthermore comprising the step of removing the stent after at least partial resorption of the treated tissues.

Clause 7. A prostatic stent characterized in that it is made of a bioresorbable or biodegradable material.

Clause 8. The stent of claim 1, wherein the biodegradable or bioresorbable material is selected from the group comprising: polylactic acid, polyglycolic acid, polycaprolactone, polydioxanone, and copolymers thereof.

Clause 9. A prostatic stent characterized by a device adapted to remove the stent without the insertion of external members into the urethra.

Clause 10. The prostatic stent of clause 9, comprising a permanent magnet adapted to cooperate with a magnetic extractor for removal of the stent from the urethra.

Clause 11. A prostatic stent, for example according to any one of the clauses 7 to 10, comprising at least one opening adapted to allow contact of the urinary flow with the wall of the urethra when the prostatic stent is implanted.

Clause 12. A prostatic stent, for example according to one or more of the clauses 7 to 11, comprising a plurality of annular elements joined to one another by a longitudinal structure.

Clause 13. The prostatic stent of clause 12, wherein the longitudinal structure has an undulated, in particular sinusoidal, shape.

Clause 14. A prostatic stent, for example according to one or more of the clauses 7 to 13, having an open loop cross section, adapted to allow a variation in the diametral dimension of the stent.

Clause 15. A prostatic stent, for example according to one or more of the clauses 7 to 14, having a structure expansible in situ.

Clause 16. A prostatic stent, for example according to one or more of the clauses 7 to 15, comprising a coating with a material selected from the group comprising: a biocompatible material, an anaesthetic material, a material having anti-inflammatory effect, a material with antibiotic effect, or combinations thereof.

Clause 17. A prostatic stent, for example according to one or more of the clauses 7 to 16, comprising anchoring structures for anchoring to the wall of the urethra applied on the outer surface of the stent.

Clause 18. A prostatic stent, for example according to one or more of the clauses 7 to 17, comprising at least one sensor adapted to detect a condition associable with the degree of resorption of treated prostatic tissue.

Clause 19. The stent of clause 18, wherein the sensor is a pressure sensor.

Clause 20. The stent of clause 18, wherein the sensor is a biochemical sensor, in particular adapted to detect one or more agents associated with an oedema or an inflammation, such as macrophages or DAMPS .

Clause 21. The stent of clause 18, wherein the sensor is biodegradable and passive.

What is claimed is:

1. A method for treating a prostate condition in a patient, the method comprising:
performing an ablative intervention on target prostate tissue of the patient;
after the ablative intervention, inserting a temporary prostatic stent into the urethra of the patient in a portion of the urethra extending through the prostate; and
maintaining the temporary prostatic stent in place for a time sufficient to maintain urethral patency during a post-treatment period while compression on the urethra caused by surrounding prostate tissue decreases as a result of the ablative intervention.

2. The method of claim 1, wherein the prostate condition comprises benign prostatic hyperplasia.

3. The method of claim 1, wherein the prostate condition comprises a prostate tumor.

4. The method of claim 1, wherein the target prostate tissue comprises hyperplastic tissue, adenomatous tissue, tumoral tissue, or a combination thereof.

5. The method of claim 1, wherein the ablative intervention comprises an energy-based ablation.

6. The method of claim 5, wherein the energy-based ablation comprises laser ablation.

7. The method of claim 5, wherein the energy-based ablation comprises radiofrequency ablation.

8. The method of claim 5, wherein the energy-based ablation comprises steam ablation, microwave ablation, ultrasound ablation, electrosurgical ablation, cryoablation, irreversible electroporation, or a combination thereof.

9. The method of claim 1, wherein the ablative intervention is performed through the urethra.

10. The method of claim 1, wherein the ablative intervention is performed transperineally or transrectally.

11. The method of claim 1, wherein the ablative intervention is performed by introducing one or more treatment members adjacent or into the target prostate tissue.

12. The method of claim 11, wherein the one or more treatment members comprise one or more energy delivery devices.

13. The method of claim 12, wherein the one or more energy delivery devices are selected from the group consisting of: optical fibers, electrodes, probes, needles, catheters, antennas, or combination thereof.

14. The method of claim 1, wherein the ablative intervention is performed using energy delivered transcutaneously or transdermally and concentrated at a focal point within the target tissue so as to induce localized thermal ablation.

15. The method of claim 1, wherein the target prostate tissue is treated in one or in a plurality of treatment volumes distributed around the urethra, in one or both lobes of the prostate.

16. The method of claim 1, wherein the target prostate tissue is treated in one or in a plurality of treatment volumes distributed in one or both lobes of the prostate.

17. The method of claim 1, wherein the ablative intervention is performed sequentially at different positions by repositioning one or more treatment members in a pull-back procedure.

18. The method of claim 1, wherein the temporary prostatic stent is bioresorbable or biodegradable.

19. The method of claim 18, wherein the temporary prostatic stent comprises polylactic acid, polyglycolic acid, polycaprolactone, polydioxanone, or a copolymer thereof.

20. The method of claim 1, further comprising removing the temporary prostatic stent after at least partial reduction of urethral compression resulting from the ablative intervention.

21. The method of claim 20, wherein removing the temporary prostatic stent comprises magnetically extracting the stent by interaction between an external magnetic extractor and a magnetic element associated with the stent.

22. The method of claim 1, wherein the temporary prostatic stent comprises one or more openings configured to permit urinary flow to contact a wall of the urethra while the stent is implanted.

23. The method of claim 1, wherein the temporary prostatic stent comprises a plurality of annular elements joined to one another by a longitudinal structure.

24. The method of claim 23, wherein the longitudinal structure is undulated.

25. The method of claim 1, wherein the temporary prostatic stent has an open-loop cross section configured to permit radial compression for insertion and radial expansion after placement in the urethra.

26. The method of claim 1, wherein the temporary prostatic stent comprises a coating including a biocompatible material, an anesthetic, an anti-inflammatory agent, an antibiotic, or a combination thereof.

27. The method of claim 1, wherein the temporary prostatic stent comprises one or more anchoring structures configured to engage a urethral wall.

28. The method of claim 1, wherein the temporary prostatic stent comprises one or more sensors configured to detect a condition associated with post-treatment tissue response.

29. The method of claim 28, wherein the one or more sensors comprise a pressure sensor or a biochemical sensor.

30. The method of claim 28, further comprising determining, based on output from the one or more sensors, when the temporary prostatic stent is to be removed.

31. The method of claim 1, wherein the temporary prostatic stent is maintained for a period of from about two weeks to about four weeks.

32. The method of claim 1, wherein the temporary prostatic stent is configured to be expandable in situ.

33. The method of claim 1, wherein the step inserting the temporary prostatic stent includes the step of introducing the temporary prostatic stent through a tubular instrument.
